(19) United States
(12) Patent Application Publication
(10) Pub. No.: US 2019/0177483 A1
(43) Pub. Date: Jun. 13, 2019

(54) METHOD FOR PRODUCING POLYIMIDES

(71) Applicant: Technische Universität Wien, Wien (AT)

(72) Inventors: Miriam Margarethe UNTERLASS, Wien (AT); Sebastian ESPANA-OROZCO, Brunn an der Wild (AT)

(21) Appl. No.: 16/325,759

(22) PCT Filed: Aug. 21, 2017
(86) PCT No.: PCT/AT2017/000058

§ 371 Date: Feb. 15, 2019

(30) Foreign Application Priority Data
Aug. 19, 2016 (AT) A 383/2016

(51) Int. Cl. C08G 73/10; C09D 179/08; C07C 65/34; C07C 211/63; C07C 55/24; C07D 307/24; B29C 48/00; B29C 43/00; D01F 6/74

(52) U.S. Cl. CPC C08G 73/1032; C08G 2101/00; C08G 73/1067; C08G 73/1082; C08G 73/1085; C09D 179/08; C07C 65/34; C07C 211/63; C07C 55/24; C07D 307/24; B29C 48/022; B29C 43/003; D01F 6/74; C08G 2150/00; C08G 73/1078

(57) ABSTRACT

A method is provided for producing polyimides by polycondensation of previously produced stoichiometric salts from polycarboxylic acids or their polyanhydrides and polyamines by heating the salts for dehydration. In the method, a) an aqueous solution of a water-soluble stoichiometric salt is produced from polycarboxylic acid and polyamine; b) the aqueous solution undergoes a processing step; and c) the salt contained in the solution is simultaneously or subsequently polycondensed, by heating to form a polyimide.

The present invention relates to a novel method for producing polyimides.

### STATE OF THE ART

Polyimides are valuable materials for various applications. They are generally synthesized by polycondensation of diamines with dianhydrides in solution, in a melt or in the solid state. Surprisingly, it was found several years ago that—despite the dehydration reaction in the course of the condensation reaction—even water can be used as a solvent for polyimide synthesis if it is conducted under so-called “hydrothermal conditions”, i.e. the reaction takes place under pressure at temperatures above 100° C. (see Hodgkin et al., “Water as a Polymerization Solvent-cyclization of Polyimides: Le Chatelier Confounded?”, Polym. Prep. (American Chemical Society, Division of Polymer Chemistry) 41, 208 (2000); and WO 99/06470). When solvents other than water are used, the term “solvothermal conditions” is used for temperatures above their boiling points.

The mechanism of this condensation reaction proceeds in two stages comprising the formation of amic acids which subsequently undergo cyclodehydration to give the corresponding imides. In 1999, Dao et al. investigated factors that significantly influence imidization reactions (Dao, Hodgkin and Morton, “Important Factors Controlling Synthesis of Imides in Water”, High Perform. Polym, 11, 205-218 (1999), “Dao 1999”) and inter alia found out that the higher the temperature of the imidization reaction, the purer the products formed.

The reason why the reaction equilibrium of this cyclization proceeding under dehydration lies, even in water as solvent, on the product side, are changed properties of the solvent under solvothermal conditions. For example, water behaves like a pseudo-organic solvent under these conditions (Hodgkin et al., supra).

Furthermore, usually a stoichiometric salt is formed from diamide and dianhydride prior to polymerization, which is usually achieved by simply mixing the monomers in water and collecting the water-insoluble and therefore precipitated salts by filtration, as is currently also described in WO 2016/032299 A1. In this case, the anhydrides undergo a hydrolysis to give the free tetracarboxylic acids, two carboxyl groups of which form an ammonium salt with one amino group each (Unterlass et al., “Mechanistic study of hydrothermal synthesis of aromatic polyimides”, Polym. Chem. 2011, 2, 1744). In the monomer salts thus obtained, which are sometimes referred to as “AH salts” (in analogy to polyamide and especially nylon synthesis), the two monomers are thus present exactly in a molar ratio of 1:1, which is why their subsequent polymerization leads to very pure polyimides. Shown below is an example of the reaction scheme of two typical aromatic monomers:

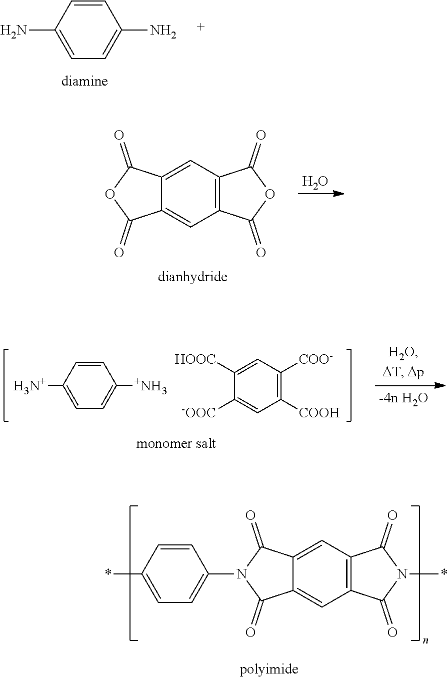

Another modern technology that has been used for several years to synthesize organic compounds and, more recently, polyimides is microwave radiation which significantly reduces reaction times and increases the selectivity of reactions (Lindstrom et al., “Microwave Assisted Organic Synthesis: a Review”, Tetrahedron 57, 9225-9283 (2001); Perreux et al., “A Tentative Rationalization of Microwave Effects in Organic Synthesis According to the Reaction Medium and Mechanistic Considerations”, Tetrahedron 57, 9199-9223 (2001)). Microwaves have also been used for the synthesis of polyimides (Lewis et al., “Accelerated Imidization Reactions using Microwave Radiation”, J. Polym. Sci., Part A: Polym. Chem. 30, 1647-1653 (1992); and U.S. Pat. No. 5,453,161).

A combination of the hydrothermal process described above and heating by means of microwave radiation is also known. On the one hand, Dao et al. (Dao, Groth and Hodgkin, “Microwave-assisted Aqueous Polyimidization Using High-throughput Techniques”, Macromol. Rapid Commun. 28, 604-607 (2007); “Dao 2007”) have, by means of serial experiments using a ternary monomer mixture of a diamine (4,4′-oxydianiline, ODA) and two dianhydrides (4,4′-(hexafluoroisopropylidene)diphthalic acid anhydride, 6-FDA, pyromellitic dianhydride, PMDA) at temperatures between 120° C. and 200° C., found that the best results are achievable at 180-200° C. if the objective is the highest possible molecular weight of the resulting statistical (block) copolymers of the following formula:

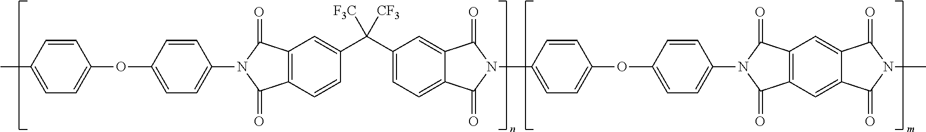

On the other hand, Brunel et al. (Brunel, Marestin, Martin and Mercier, “Waterborne Polyimides via Microwave-assisted Polymerization”, High Perform. Polym. 22, 82-94 (2010)) confirmed once again only a few years ago, by using a binary polyimide of ODA and 4,4′-(4,4′-isopropylidenediphenoxy)bis(phthalic anhydride) (bisphenol A dianhydride, BPADA)

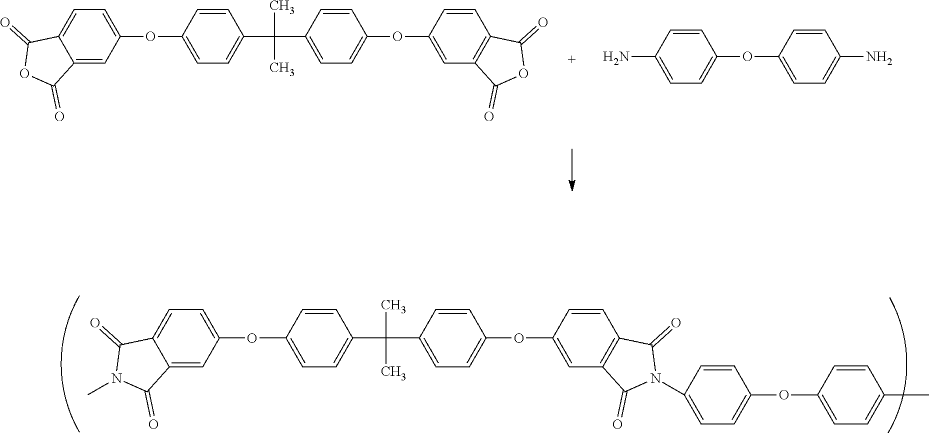

that the use of microwaves allows a significant reduction of reaction times, i.e. from 4 to 12 h down to only 5 to 10 min. The turnovers achieved in this short time, however, are extremely low at only about 20%. However, in both cases, no crystalline products could be obtained.

US 2008/300360 A1 discloses an alternative process using water as solvent to prepare water-based coating solutions. However, this process does not start with AH salts, but first produces prepolymers, i.e. low-molecular oligomers, from previously dewatered polyamides and polyanhydrides in the presence of a defined content of e.g. 5 to 60 mole percent of monoanhydrides as terminators, from which subsequently suspensions or—after grinding to obtain smaller particle sizes—colloidal solutions are formed in water, which can be used for coating surfaces. In order to prepare stable suspensions and colloidal solutions, it is also preferable to add suspension stabilizers to the prepolymers obtained, which are characterized as being insoluble in water. These aqueous systems are processed into coatings, particularly laminates, by application to a surface, evaporation of the water, and heating to initiate polycondensation of the prepolymers to macromolecules having molecular weights of at least 10,000.

With respect to the solvothermal synthesis of polyimides, the inventors of the present subject matter have recently found that crystalline polyimides may also be obtained under hydrothermal conditions when either the solvent is heated to solvothermal conditions and only then the monomers are added to initiate the reaction, or the monomers are mixed with the solvent and the mixture is heated to solvothermal conditions within 5 minutes, the reaction temperature being maintained during the polymerization at the polymerization temperature of the monomers in the solid state (B. Baumgartner, M. J. Bojdys, P. Skrinjar and M. M. Unterlass, “Design Strategies in Hydrothermal Polymerization of Polyimides”, Macromol. Chem. Phys. 217, 485-500 (2016)].

Despite these advantageous recent developments, the high expenditures regarding apparatuses and energy to achieve hydrothermal conditions so that water can be used as a solvent for the polycondensation reaction continues to be a considerable disadvantage. Against this background, the object of the present invention was to provide a method by which this disadvantage can be overcome.

### DISCLOSURE OF THE INVENTION

The present invention solves this problem by providing a method for producing polyimides from polycarboxylic acids or their polyanhydrides and polyamines by polycondensation of previously prepared stoichiometric salts by heating the salts for dehydration, which method is characterized in that

a) an aqueous solution of a water-soluble stoichiometric salt of polycarboxylic acid and polyamine is prepared;
b) the aqueous solution is subjected to a processing step; and
c) the salt contained in the coating is simultaneously or subsequently polycondensed by heating to give a polyimide.

This invention is based on the most surprising finding by the inventors that some monomer salts of polycarboxylic acid and polyamide are—contrary to all other such salts known—water-soluble and thus do not precipitate from an aqueous solution upon mixing of the monomers. Due to this unique property, it is possible to directly subject such an aqueous solution of the monomer salts to a processing step and simultaneously or sequentially subject them to polycondensation by heating—preferably to a temperature Tp higher than the solid state polymerization temperature of the salt in order to guarantee complete conversion of the polycondensation reaction.

In preferred embodiments, the aqueous solution is applied to a support or a substrate to obtain a coating, and the resulting coating is polycondensed by heating. In this case, the coating is preferably dried before the polycondensation in step c), so that a film or a solid film is formed on the substrate, which is easier to handle than the moist coating.

In alternative preferred embodiments, the aqueous solution is foamed in the processing step b), after which the resulting foam is cured by polycondensation in the subsequent step c) to obtain a cured polyimide foam. In still other preferred embodiments, the aqueous solution is fed to a nozzle heated to a temperature above Tp in the processing step b), where the stoichiometric salt is simultaneously cured by polycondensation and the resulting polyimide is forced through the nozzle opening(s) in step c) to obtain a molded polyimide product. The polyimide thus obtained may be wet-spun, press-molded or extruded into filaments, depending on the particular nozzle used and the associated molding device. Wet-spun filaments are subsequently preferably wound onto spools to obtain polyimide fibers.

The water-soluble stoichiometric salt is prepared in step a) preferably by mixing stoichiometric amounts of a polycarboxylic acid or its polyanhydride and polyamine in water or in an aqueous solvent mixture. Furthermore, the water-soluble stoichiometric salt is preferably precipitated by the addition of at least one organic solvent for intermediate storage prior to polycondensation. The organic solvent used is therefore not particularly limited as long as it sufficiently lowers the solubility of the stoichiometric salt in the aqueous solvent mixture formed to bring about its precipitation, provided that it is itself a nonsolvent for the stoichiometric salt. Both water-miscible and water-immiscible solvents can be used for this purpose, for example, alcohols such as methanol or other lower alcohols, ethers such as THF, acetone, etc.

In preferred embodiments of the present invention, as already mentioned, a film is drawn from the aqueous solution of the water-soluble stoichiometric salt on a substrate in processing step b) in order to optionally form a composite material after the subsequent polycondensation of the salt to give the corresponding polyimide or to obtain a polyimide film after pulling off the cured polyimide film from the substrate.

According to the present invention, a water-soluble stoichiometric salt is preferably prepared from a tetracarboxylic acid and a diamine and polycondensed because these are the most common starting materials in polyimide production. However, combinations of other polyvalent amines and/or polycarboxylic acids or their anhydrides are, of course, also possible, e.g. the preparation of stoichiometric salts of triamine and dianhydride, diamine and trianhydride, triamine and trianhydride, etc., as long as these monomers form water-soluble salts in the corresponding stoichiometric composition.

It is particularly preferred that the tetracarboxylic acid is selected from benzophenone-tetracarboxylic acid, tetrahydrofurantetracarboxylic acid, butanetetracarboxylic acid and their dianhydrides, since the inventors have already found water-soluble stoichiometric salts of these polycarboxylic acids. For the same reason, the diamine is preferably selected from benzenedimethaneamine and ethylenediamine. In particular, the stoichiometric salts are selected from 1,3-benzenedimethaneammoniumdihydrogen-3,3′,4,4′-benzophenonetetracarboxylate (1), 1,3-benzenedimethaneammonium-dihydrogen-1,2,3,4-butanetetracarboxylate (2), 1,3-benzenedimethaneammonium-dihydrogentetrahydrofuran-2,3,4,5-tetracarboxylate (3), ethane-1,2-diammonium-dihydrogen-3,3′,4,4′-benzophenonetetracarboxylate (4), ethane-1,2-diammonium-dihydrogentetrahydrofuran-2,3,4,5-tetracarboxylate (5) and hydrates thereof, with which excellent results have already been achieved in the inventive method, wherein the hydrates are easier to produce in crystalline form than the anhydrous ammonium salts.

Since these salts are all novel, i.e. have been synthesized and characterized for the first time by the inventors, which at the same time is the reason why their solubility in water has not yet been discovered, the present invention in a second aspect also provides these specific compounds, namely:

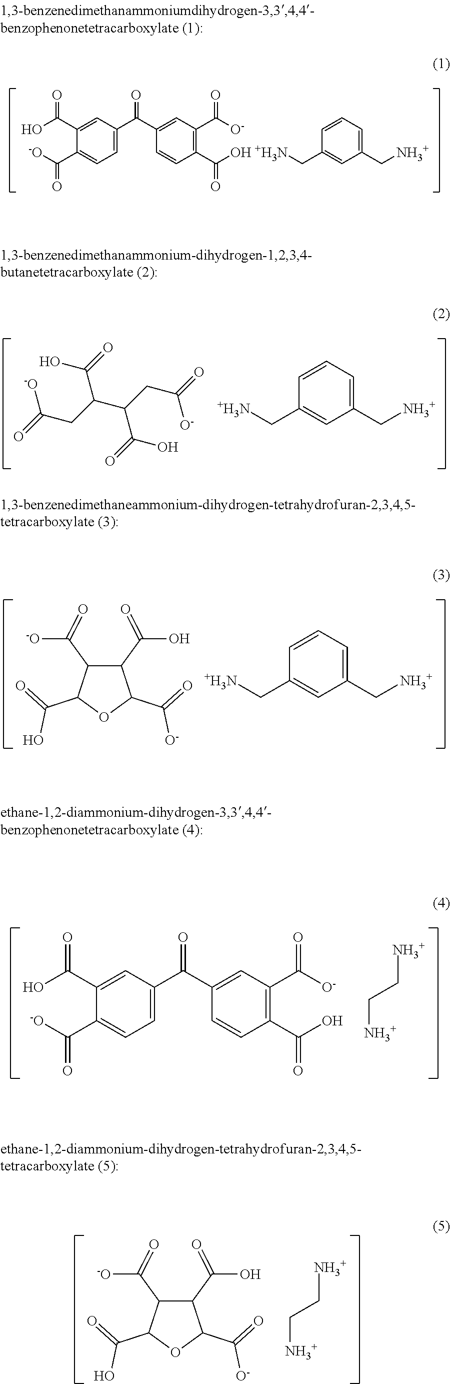

Furthermore, the present invention also provides two novel polyimides synthesized—from the corresponding water-soluble stoichiometric salts (3) and (5)—and characterized for the first time by the inventors, namely:

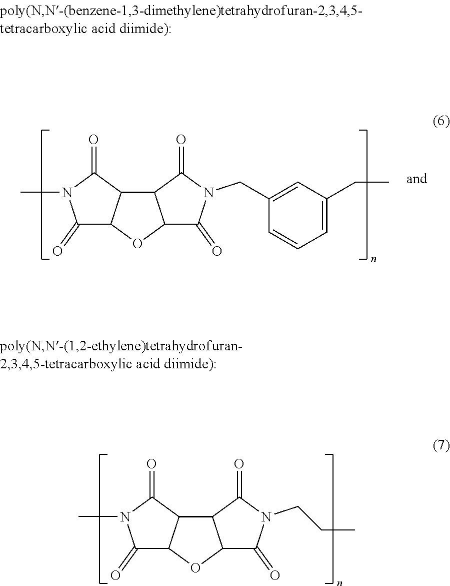

wherein each n≥2.

### BRIEF DESCRIPTION OF THE DRAWINGS

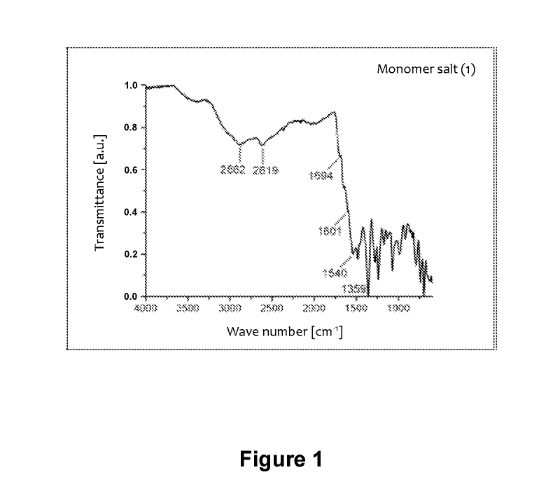

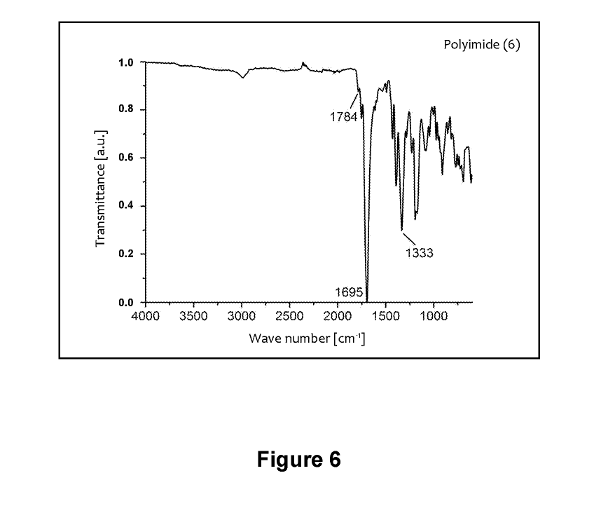

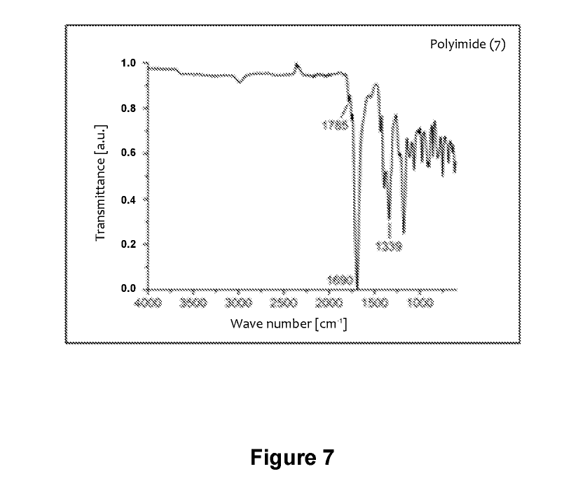

Below, the present invention will be further described by way of non-limiting examples with reference to the accompanying drawings, wherein FIGS. 1 to 5 are IR spectra of the water-soluble stoichiometric monomer salts obtained in Examples 1, 4, 6, 7 and 8, and FIGS. 6 and 7 are IR spectra of the polyimides obtained in Examples 12 and 13.

### EXAMPLES

All reagents used in the experiments below are commercially available and were used without further purification. The IR spectra shown in the accompanying drawings were recorded by means of FT-IR ATR spectroscopy on a Bruker Tensor 27 and ¹H NMR spectra were recorded on an Avance 250, also from Bruker. In the following, “Tp” refers to the solid state polymerization temperature of the obtained stoichiometric salts.

### Example 1

### Preparation of 1,3-benzenedimethaneammonium-dihydrogen-3,3′,4,4′-benzophenonetetracarboxylate (1)

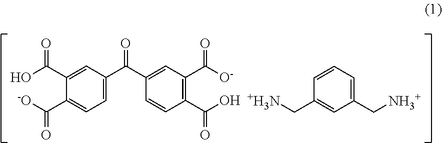

150 mg (0.47 mmol) of 3,3′,4,4′-benzophenontetracarboxylic acid dianhydride were suspended in 10 ml of dist. Wasser, and 61.4 μl (0.47 mmol) of 1,3-benzenedimethaneamine were added, forming a clear, yellowish solution. After stirring for 30 minutes, the water was removed on a rotary evaporator in a water bath (50° C.) under a vacuum of approximately 60 mbar and the residue was dried under high vacuum. The title compound was quantitatively obtained as a colorless amorphous solid, the IR spectrum of which is shown in FIG. 1. The solid is highly hygroscopic and gradually deliquesces in the air to a yellow liquid phase of the tetrahydrate.

Tp.: 151° C. (DSC) or 161° C. (TGA) (heating rate: 10 K/min)

¹H NMR (250 MHz, DMSO-d₆) δ: 8.51 (d, 2H, ar), 8.32 (d, 2H, ar), 7.85 (q, 2H, ar), 7.45 (m, 4H, ar), 4.01 (s, 4H, aliph).

IR (cm⁻¹): 2882, 2619, 1694, 1601, 1540, 1359.

### Example 2

### Precipitation of the Tetrahydrate of (1) by Addition of a Solvent

The approach of Example 1 was repeated with the addition of 30 ml of THF to the resulting aqueous solution of (1) (here: in 5 ml of dist. water), forming a precipitate in the form of a white turbidity. The mixture was allowed to stand overnight, during which time the initial precipitate forming a yellow-colored second liquid phase, which crystallized after another 24 hours of rest in the form of colorless crystals.

The data correlated with those of Example 1, except for the presence of water.

### Example 3

### Preparation of (1) as an Aqueous Solution and Coating of a Surface

Example 1 was repeated, wherein instead of isolating the stoichiometric salt (1) by evaporating the water, the aqueous solution was used for coating a glass plate. For this purpose, a few drops of the solution were dropped onto a glass plate and allowed to dry in air. Subsequently, polycondensation to poly(N,N′-(benzene-1,3-dimethylene)-benzophenone-3,3′,4,4′-tetracarboxylic acid diimide) was carried out in a vacuum oven maintained at 200° C. overnight. The IR spectrum of the polyimide thus obtained corresponded to that of the product known from the literature.

### Example 4

### Preparation of 1,3-benzenedimethanammonium-dihydrogen-1,2,3,4-butanetetracarboxylate (2)

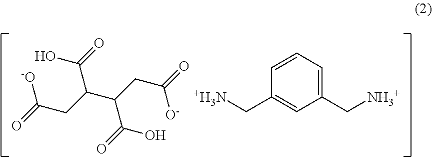

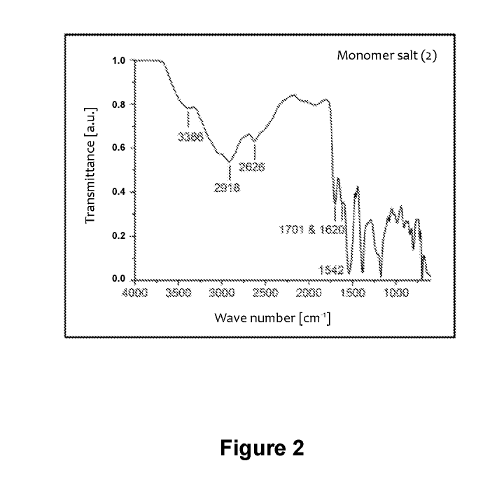

150 mg (0.64 mmol) of 1,2,3,4-butanetetracarboxylic acid were dissolved in 10 ml of dist. water, and 84.5 μl (0.64 mmol) of 1,3-benzenedimethanamine were added at once, after which the reaction mixture was shaken until a clear solution formed. Work-up and drying were carried out analogously to Example 1, and the title compound was obtained in a quantitative yield as a hygroscopic, colorless, amorphous solid, the IR spectrum of which is shown in FIG. 2.

Tp.: 151° C. (TGA)

¹H NMR (250 MHz, D₂O) δ: 7.5 (m, 4H, ar), 4.2 (s, 4H, aliph), 2.9 (m, 2H, aliph), 2.6 (m, 2H, aliph), 2.4 (m, 2H, aliph).

IR (cm⁻¹): 3386, 2918, 2626, 1701, 1620, 1542.

### Example 5

### Precipitation of the Tetrahydrate of (2) by Addition of a Solvent

The approach of Example 4 was repeated with the addition of 30 ml of THF to the resulting aqueous solution of (2) (here: in 5 ml of dist. water), which formed a precipitate in the form of a white turbidity. The mixture was allowed to stand overnight, during which time the initial precipitate formed a yellow-colored second liquid phase, which crystallized after another 24 hours of rest in the form of colorless crystals.

The data correlated with those of Example 4, except for the presence of water.

### Example 6

### Preparation of 1,3-benzenedimethanammoniumdihydrogentetrahydrofuran-2,3,4,5-tetracarboxylate (3)

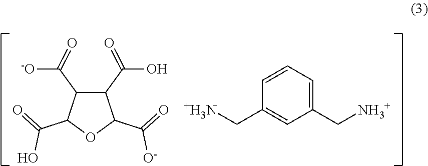

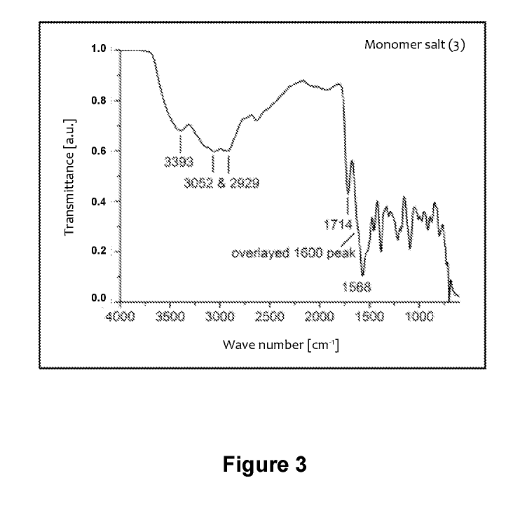

150 mg (0.60 mmol) of tetrahydrofuran-2,3,4,5-tetracarboxylic acid were dissolved in 10 ml of dist. water, and 79.8 μl (0.60 mmol) of 1,3-benzenedimethanamine were added at once, after which the reaction mixture was shaken until a clear solution formed. Work-up and drying were carried out analogously to Example 1, giving a hygroscopic, colorless, amorphous solid in quantitative yield, the IR spectrum of which is shown in FIG. 3.

Mp.: 62° C. (DSC)

Tp.: 144° C. (DSC) or 151° C. (TGA) (heating rate: 10 K/min)

¹H NMR (250 MHz, DMSO-d₆) δ: 7.5 (s, 1H, ar), 7.4 (m, 3H, ar), 4.4 (m, 2H, aliph), 4.0 (s, 4H, aliph), 3.0 (m, 2H, aliph).

IR (cm⁻¹): 3393, 3052, 2929, 1714, 1600, 1568.

### Example 7

### Preparation of ethane-1,2-diammoniumdihydrogen-3,3′,4,4′-benzophenonetetracarboxylate (4)

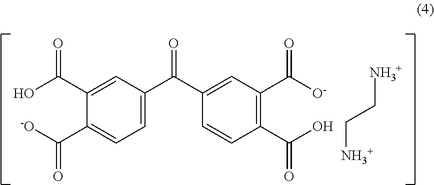

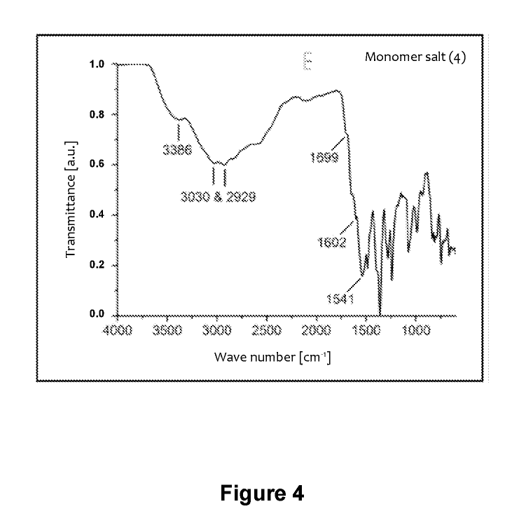

150 mg (0.47 mmol) of 3,3′,4,4′-benzophenontetracarboxylic acid dianhydride were dissolved in 10 ml of dist. water, and 31.1 μl (0.47 mmol) 1,2-ethylenediamine were added at once, after which the reaction mixture was shaken until a clear solution formed. Work-up and drying were carried out analogously to Example 1, giving a hygroscopic, colorless, amorphous solid in quantitative yield, the IR spectrum of which is shown in FIG. 4.

Tp.: 128° C. (DSC) or 149° C. (TGA) (heating rate: 10 K/min)

¹H NMR (250 MHz, D₂O) δ: 8.1 (d, 2H, ar), 7.9 (q, 2H, ar), 7.7 (d, 2H, ar), 3.4 (s, 4H, aliph).

IR (cm⁻¹): 3386, 3030, 2929, 1699, 1602, 1541.

### Example 8

### Preparation of ethane-1,2-diammonium-dihydrogen-tetrahydrofuran-2,3,4,5-tetracarboxylate (5)

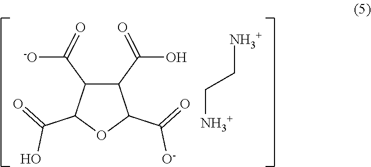

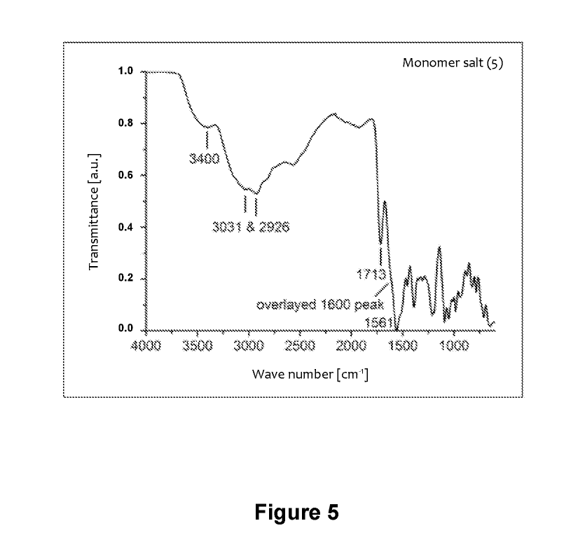

150 mg (0.60 mmol) of tetrahydrofuran-2,3,4,5-tetracarboxylic acid were dissolved in 10 ml of dist. water, and 40.4 μl (0.60 mmol) 1,2-ethylenediamine were added at once, after which the reaction mixture was shaken until a clear solution formed. Work-up and drying were carried out analogously to Example 1, giving a hygroscopic, colorless, amorphous solid in quantitative yield, the IR spectrum of which is shown in FIG. 5.

Tp.: 128° C. (DSC) or 149° C. (TGA) (heating rate: 10 K/min)

¹H NMR (250 MHz, D₂O) δ: 4.8 (m, 2H, aliph), 3.5 (m, 2H, aliph), 3.4 (s, 4H, aliph).

IR (cm⁻¹): 3400, 3031, 2926, 1713, 1600, 1561.

### Examples 9 to 13

### Coating and Polycondensation

The approaches of Examples 1, 4, 6, 7 and 8 were repeated using the aqueous solutions for coating glass plates in a way similar to Example 3 instead of isolating the stoichiometric salts. For this purpose, 5 to 10 ml of the respective aqueous solution were applied to a glass plate, which was then heated in an oven to 250° C. at a heating rate of 5 K/min and subsequently maintained at this temperature for 30 min.

The polyimides thus obtained were three products known from the literature, namely in Example 9 (with the stoichiometric salt obtained analogously to Example 1):

poly(N,N′-(benzene-1,3-dimethylene)benzophenone-3,3′,4,4′-tetracarboxylic acid diimide);
in Example 10 (with the stoichiometric salt obtained analogously to Example 4): poly(N,N′-(benzene-1,3-dimethylene)butane-1,2,3,4-tetracarboxylic acid diimide), and
in Example 1 (with the stoichiometric salt obtained analogously to Example 7): poly(N,N′-(1,2-ethylene)benzophenone-3,3′,4,4′-tetracarboxylic acid diimide); and two previously unknown polyimides, namely:
in Example 12 (with the stoichiometric salt obtained analogously to Example 6): poly(N,N′-(benzene-1,3-dimethylene)tetrahydrofuran-2,3,4,5-tetracarboxylic acid diimide) (6); and
in Example 13 (with the stoichiometric salt obtained analogously to Example 8): poly(N,N′-(1,2-ethylene)tetrahydrofuran-2,3,4,5-tetracarboxylic acid diimide) (7).

These polyimides were, inter alia, analyzed by means of IR spectroscopy. The IR spectra of the products of Examples 9 to 11 corresponded to those of the polyimides known from literature.

Regarding the novel polyimides (6) and (7), the decomposition points were determined in addition to the IR spectra shown in FIGS. 6 and 7, and furthermore a ¹H NMR spectrum of polyimide (6) was recorded (for polyimide (7) impossible because it was insoluble in the solvents tested), providing the following data.

### Poly(N,N′-(benzene-1,3-dimethylene)tetrahydrofuran-2,3,4,5-tetracarboxylic acid diimide) (6)

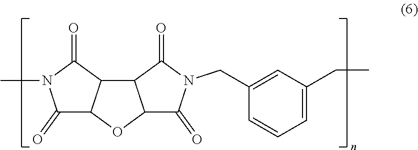

Mp.: 456° C. (dec.) (TGA, heating rate: 10 K/min)

¹H NMR (250 MHz, DMSO-d₆) δ: 7.3 (s, 1H, ar), 7.1 (d, 3H, ar), 5.2 (t, 1H, aliph), 4.7 (s, 1H, aliph), 4.6 (s, 2H, aliph), 4.5 (s, 2H, aliph), 3.9 (s, 2H, aliph).

IR (cm⁻¹): 1784, 1695, 1333.

### Poly(N,N′-(1,2-ethylene)tetrahydrofuran-2,3,4,5-tetracarboxylic acid diimide) (7)

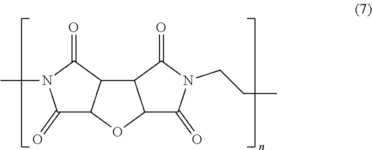

Mp.: 438° C. (dec.) (TGA, heating rate: 10 K/min)

IR (cm⁻¹): 1785, 1690, 1339.

The present invention thus provides a process by which polyimide coatings can be prepared from aqueous solutions of the stoichiometric monomer salts, which is a considerable advantage over prior art because it limits or even completely avoids the use of expensive solvents that can only be removed with enormous energy input.

In addition, the invention shall not be limited to the five monomer combinations disclosed herein, since a person skilled in the art—who now has the knowledge that such water-soluble monomer salts actually exist—can easily, by means of simple routine experiments, determine other combinations of polycarboxylic acid or polyanhydride and polyamines that result in a water-soluble stoichiometric salt.

1.-16. (canceled)

17. 1,3-Benzenedimethaneammonium-dihydrogen-3,3′,4,4′-benzophenonetetracarboxylate (1):

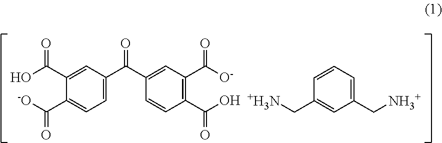

18. 1,3-Benzenedimethaneammonium-dihydrogen-1,2,3,4-butanetetracarboxylate (2):

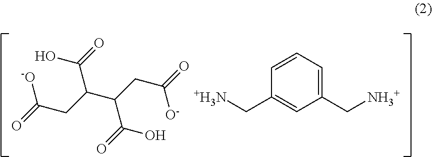

19. 1,3-Benzenedimethaneammonium-dihydrogen-tetrahydrofuran-2,3,4,5-tetracarboxylate (3):

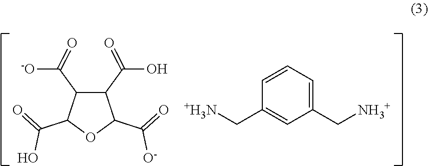

20. Ethane-1,2-diammonium-dihydrogen-3,3′,4,4′-benzophenonetetracarboxylate (4):

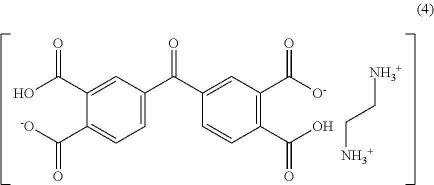

21. Ethane-1,2-diammonium-dihydrogen-tetrahydrofuran-2,3,4,5-tetracarboxylate (5):

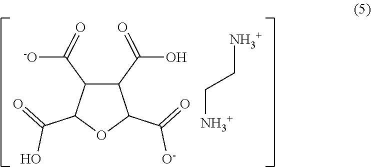

22. Poly(N,N′-(benzene-1,3-dimethylene)tetrahydrofuran-2,3,4,5-tetracarboxylic acid diimide) (6): 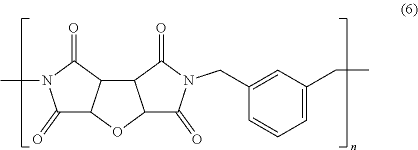
wherein n≥2.

23. Poly(N,N′-(1,2-ethylene)tetrahydrofuran-2,3,4,5-tetracarboxylic acid diimide) (7): 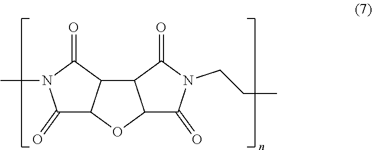
wherein n≥2.

24. A method for producing polyimides from polycarboxylic acids or their polyanhydrides and polyamines by polycondensation of previously prepared stoichiometric salts by heating the salts for dehydration, wherein:
a) an aqueous solution of a water-soluble stoichiometric salt of polycarboxylic acid and polyamine is prepared;
b) the aqueous solution is subjected to a processing step; and
c) the salt contained in the coating is simultaneously or subsequently polycondensed by heating to give a polyimide.

25. The method of claim 24, wherein heating in step c) is performed at a temperature Tp higher than the polymerization temperature of the salt.

26. The method of claim 24, wherein in processing step b), a substrate is coated with the aqueous solution in order to obtain a coating that is cured by polycondensation in the subsequent step c).

27. The method of claim 26, wherein the coating obtained in processing step b) is dried prior to polycondensation in step c).

28. The method of claim 27, wherein in processing step b), a film is drawn from the aqueous solution of the water-soluble stoichiometric salt on the substrate.

29. The method of claim 24, wherein in processing step b), the aqueous solution is foamed to obtain a foam that is cured by polycondensation in the subsequent step c) to obtain a cured polyimide foam.

30. The method of claim 24, wherein in processing step b), the aqueous solution is fed to a nozzle heated to a temperature higher than Tp, where the stoichiometric salt is simultaneously cured by polycondensation, and in step c), the resulting polyimide is forced through nozzle opening(s) to obtain a molded polyimide product.

31. The method of claim 30, wherein the obtained polyimide is wet-spun, press-molded or extruded.

32. The method of claim 24, wherein in step a), the water-soluble stoichiometric salt is prepared by mixing stoichiometric amounts of a polycarboxylic acid or its polyanhydride and polyamine in water or in an aqueous solvent mixture.

33. The method of claim 32, wherein the water-soluble stoichiometric salt is precipitated by the addition of an organic solvent for intermediate storage prior to polycondensation.

34. The method of claim 24, wherein a water-soluble stoichiometric salt is prepared from a tetracarboxylic acid and a diamine and polycondensed.

35. The method of claim 34, wherein the tetracarboxylic acid is selected from benzo-phenonetetracarboxylic acid, tetrahydrofurantetracarboxylic acid, and butanetetracarboxylic acid.

36. The method of claim 34, wherein the diamine is selected from benzenedimethaneamine and ethylenediamine.

37. The method of claim 36, wherein the stoichiometric salt is selected from 1,3-benzenedimethaneammonium-dihydrogen-3,3′,4,4′-benzophenonetetracarboxylate (1), 1,3-benzenedimethaneammonium-dihydrogen-1,2,3,4-butanetetracarboxylate (2), 1,3-benzenedimethaneammonium-dihydrogen-tetrahydrofuran-2,3,4,5-tetracarboxylate (3), ethane-1,2-diammonium-dihydrogen-3,3′,4,4′-benzophenonetetracarboxylate (4), and ethane-1,2-diammonium-dihydrogen-tetrahydrofuran-2,3,4,5-tetracarboxylate (5).

38. The method of claim 37, wherein 1,3-benzenedimethaneammonium-dihydrogen-tetrahydrofuran-2,3,4,5-tetracarboxylate (3) is used as the stoichiometric salt and the polyimide poly(N,N′-(benzene-1,3-dimethylene)tetrahydrofuran-2,3,4,5-tetracarboxylic acid diimide) (6) is prepared by polycondensation.

39. The method of claim 37, wherein ethane-1,2-diammonium-dihydrogen-tetrahydrofuran-2,3,4,5-tetracarboxylate (5) is used as the stoichiometric salt and the polyimide poly(N,N′-(1,2-ethylene)tetrahydrofuran-2,3,4,5-tetracarboxylic acid diimide) (7) is prepared by polycondensation.
